(19) United States
(12) Patent Application Publication
(10) Pub. No.: US 2010/0261228 A1
(43) Pub. Date: Oct. 14, 2010

(54) MULTIPLEXED SITES FOR POLYMER SYNTHESIS

(75) Inventors: Morteza Gharib, San Marino, CA (US); Derek Rinderknecht, Arcadia, CA (US); Randy Keen, Laguna Hills, CA (US)

Correspondence Address: KNOBBE MARTENS OLSON & BEAR LLP, 2040 MAIN STREET, FOURTEENTH FLOOR, IRVINE, CA, 92614, US

(73) Assignee: California Institute of Technology, Pasadena, CA (US)

(21) Appl. No.: 12/756,886

(22) Filed: Apr. 8, 2010

Related U.S. Application Data
(60) Provisional application No. 61/168,170, filed on Apr. 9, 2009.
(60) Provisional application No. 61/168,730, filed on Apr. 13, 2009.

(51) Int. Cl. C07H 21/04; C12P 19/34; B01J 19/12; C12M 1/42

(52) U.S. Cl. 435/91.1; 536/25.3; 422/134; 435/299.1

(57) ABSTRACT

Disclosed herein are methods, devices, and other components for synthesizing polymers. In some embodiments, numerous sites can be multiplexed together to allow for effective nucleic acid synthesis.

### CROSS-REFERENCE TO RELATED APPLICATIONS

The present application claims priority to U.S. provisional application No. 61/168,730, filed Apr. 13, 2009 and U.S. provisional application No. 61/168,170, filed Apr. 9, 2009, both of which are incorporated by reference in their entireties.

### BACKGROUND OF THE INVENTION

1. Field of the Invention

The embodiments described herein relate to the field of polymer synthesis.

2. Description of the Related Art

A variety of methods are available for synthesizing nucleic acids. Such methods include those described in U.S. Pat. Pub. 2008/0318806, published Dec. 25, 2008.

### SUMMARY OF THE INVENTION

In some embodiments, devices and methods are provided that allow for multiplexing various sites together so as to allow the synthesis of nucleic acids. In some embodiments, the multiplexing allows for a larger number of subreactions (desired building fragment synthesis and or combination of such fragments) to occur in a relatively small size or volume, while producing relatively long lengths of a polymer. In some embodiments, the polymer comprises a nucleic acid.

In some embodiments, a method for synthesis of a desired polymer is provided. In some embodiments, the method comprises, consists of, or consists essentially of (a) providing a first reaction site comprising a first monomer attached to a surface of the reaction site; (b) selectively irradiating the first reaction site, thereby coupling an additional monomer to the first monomer; (c) repeating said irradiating until a desired single stranded fragment has been synthesized, wherein the desired single stranded fragment is created while the first monomer is attached to the surface; (d) separating the desired single stranded fragment from the surface; (e) repeating processes (a) through (d) as desired to create a desired number of desired single stranded fragments; (f) combining the first desired single stranded fragment with a specified length and sequence with a second desired single stranded fragment with a specified length and sequence so as to create a first subpolymer; (g) storing the first subpolymer in a first storage site; (h) repeating processes (a) through (f) so as to make a second subpolymer and storing said second subpolymer in a second storage site; and (i) combining the first and second subpolymers so as to form a desired polymer. In some embodiments, the irradiating process results in a photo generated reagent and wherein the polymer comprises a nucleic acid. In some embodiments, the nucleic acid comprises a DNA. In some embodiments, process (d) involves irradiating the desired single stranded fragment with a wavelength of light that disrupts the attachment of the first monomer to the surface of the reaction site without inadvertently adding an additional monomer to the desired single stranded fragment. In some embodiments, the first and second desired single stranded fragments are combined in a combination site. In some embodiments, the first reaction site comprises a chamber that is fluidly isolated from the second reaction site. In some embodiments, process (f) comprises combining the first desired single stranded fragment with the second desired single stranded fragment so as to create a first-level fragment. In some embodiments, the first-level fragment comprises a double-stranded nucleic acid, a multi-stranded fragment, or a double-stranded and a multi-stranded fragment. In some embodiments, process (f) comprises combining i) the first desired single stranded fragment with ii) the second desired single stranded fragment, and iii) a third desired single stranded fragment to form a first-level fragment. In some embodiments, the first-level fragment is chemical or enzyme ligated forming a first ligated first-level fragment. In some embodiments, any of the above process (e.g., the above two processes) are repeated so as to form a second ligated first-level fragment, and wherein the first and second ligated first-level fragments are combined so as to allow the first and second ligated first-level fragments to hybridize to one another, forming a second-level fragment. In some embodiments, the method further comprises the process of performing a ligation reaction thereby forming a ligated second-level fragment. In some embodiments, the method further comprises the step of repeating the above two processes a desired number of times to form one or more additional ligated second-level fragments. In some embodiments, the method further comprises combining one or more ligated second-level fragments to create a third-level fragment and ligating the third-level fragment. In some embodiments, the method further comprises repeating the above process one or more times to create one or more ligated third-level fragments. In some embodiments, the method further comprises combining the one or more ligated third-level fragments to create a fourth-level fragment. In some embodiments, the method further comprises repeating the method of the above process one or more times to create one or more ligated fourth-level fragments. In some embodiments, the method further comprises combining the one or more ligated fourth-level fragments to create the subpolymer. In some embodiments, the first and second desired single-stranded fragments hybridize to one another when forming the first-level fragment. In some embodiments, a third, fourth, fifth, and sixth single stranded fragments are combined and hybridize to one another to form the first-level fragment. In some embodiments, the desired polymer comprises a programmed, specified, and/or random nucleotide sequence. In some embodiments, the desired single stranded fragment comprises a programmed, specified, and/or random nucleotide sequence. In some embodiments, selectively irradiating the first reaction site, thereby coupling a monomer to the first monomer comprises a wavelength in the 100 to 1000 nm range. In some embodiments, process (d) involves a wavelength in the 100 to 1000 nm range, wherein the wavelength in process (d) may or may not overlap with the wavelength in processes (b) and/or (c). In some embodiments, the method further comprises the process of filtering so that an undesired single stranded fragment that has been synthesized in a reaction site is not contained within the subpolymer. In some embodiments, the filtering occurs after process (d). In some embodiments, the filtering process comprises a size exclusion filter such that single stranded fragments that are shorter than the undesired single stranded fragment are excluded or removed from the system prior to their combination with the desired single stranded fragment.

In some embodiments a device for parallel and serial polymer creation is provided comprising a first reaction site; a second reaction site, wherein the first and second reaction sites comprise a surface that allows for the attachment of a polymer to said surface in each reaction site, wherein the at least two reaction sites are effectively optically transparent to a first set of wavelengths of light that allows for the creation of a photo-generated reagent, wherein the first and second reaction sites are effectively transparent to a second set of wavelengths of light that allows for the cleavage of a bond that connects a nucleic acid to said surface; a first-level combination sites, wherein the first and second reaction sites are in fluid communication with the first-level combination site, wherein the fluid communication allows for the combination of a sample from the first reaction site with a sample from a second reaction site, wherein the first-level combination site is associated with a heating element that controls the temperature of the first-level combination site so as to control nucleic acid annealing and ligation; and one or more storage sites, controllably fluidly connected to the first-level combination site, wherein the one or more storage sites can be controllably fluidly sealed from the first-level combination site. In some embodiments, the device further comprises a nucleic acid that is at least 500 nucleotides in length. In some embodiments, the device further comprises a nucleic acid that is at least 10,000 nucleotides in length. In some embodiments, the device further comprises one or more fluid inlets for adding liquid to the reaction site. In some embodiments, the device further comprises one or more fluid inlets for adding liquid to the first-level combination site. In some embodiments, the device further comprises a light directing apparatus for selectively directing light to the first reaction site while avoiding directing light to the second reaction site. In some embodiments, the light directing apparatus comprises a panel of articulating mirrors. In some embodiments, first-level combination site comprises a channel that connects the at least two reaction sites to one another. In some embodiments, the device further comprises a second-level combination site, wherein at least two first-level combination sites are fluidly connected to the second-level combination site. In some embodiments, the second-level combination site is associated with a heating element that controls the temperature of the second-level combination site so as to control nucleic acid annealing in the second-level combination site. In some embodiments, the device further comprises a third-level combination site, wherein at least two second-level combination sites are fluidly connected to the third-level combination site. In some embodiments, the third-level combination site is associated with a heating element that controls the temperature of the third-level combination site so as to control nucleic acid annealing in the third-level combination site. In some embodiments, the device further comprises a fourth-level combination site, wherein at least two third-level combination sites are fluidly connected the fourth-level combination site. In some embodiments, the fourth-level combination site is associated with a heating element that controls the temperature of the fourth-level combination site so as to control nucleic acid annealing in the fourth-level combination site.

In some embodiments, the device comprises at least 7776 reaction sites; at least 216 first-level combination sites; at least 36 second-level combination sites; at least 6 third level combination sites; and at least 6 storage sites. In some embodiments, the device further comprises an output.

In some embodiments, the device further comprises one or more fluid inlets and outlets for adding and removing liquid to the reaction site. In some embodiments, the device further comprises one or more fluid inlets and outlets for adding and removing liquid to the first-level combination site. In some embodiments, the device further comprises a light directing apparatus for selectively directing light to the first reaction site while avoiding directing light to the second reaction site. In some embodiments, the light directing apparatus comprises a panel of articulating mirrors. In some embodiments, first-level combination site comprises a channel that connects the at least two reaction sites to one another. In some embodiments, the device further comprises one or more fluid inlets and outlets for adding and removing liquid to the second-level combination site. In some embodiments, the device further comprises a second-level combination site, wherein at least two first-level combination sites are fluidly connected to the second-level combination site. In some embodiments, the second-level combination site is associated with a heating element that controls the temperature of the second-level combination site so as to control nucleic acid annealing and ligation in the second-level combination site. In some embodiments, the device further comprises a third-level combination site, wherein at least two second-level combination sites are fluidly connected to the third-level combination site. In some embodiments, the third-level combination site is associated with a heating element that controls the temperature of the third-level combination site so as to control nucleic acid annealing in the third-level combination site. In some embodiments, the device further comprises a fourth-level combination site, wherein at least two third-level combination sites are fluidly connected the fourth-level combination site. In some embodiments, the fourth-level combination site is associated with a heating element that controls the temperature of the fourth-level combination site so as to control nucleic acid annealing in the fourth-level combination site. In some embodiments, the device comprises at least 7776 reaction sites; at least 216 first-level combination sites; at least 36 second-level combination sites; at least 6 third level combination sites; and at least 6 storage sites. In some embodiments, the device further comprises an output.

In some embodiments, a device for polymer synthesis is provided comprising a first reaction site and a second reaction site, fluidly connected to a combination site, wherein the combination site is fluidly connected to a first storage site; a third reaction site and a fourth reaction site, fluidly connected to a combination site, wherein the combination site is fluidly connected to a second storage site; a second combination site, fluidly connected to the first storage site and the second storage site; and an output, wherein the output allows a fluid in the second combination site to exit the device.

In some embodiments, a system of DNA synthesis is provided where multiplexing is used to collapse 2 or more reaction site into a single channel.

In some embodiments, a method of DNA synthesis is provided that combines both photogenerated acid for the assembly of ssDNA and a photocleavage step.

In some embodiments, methods employing branching channels along a main fluidic channel to assemble segments of dsDNA greater than 100 bp in length are provided.

In some embodiments, a device for polymer synthesis is provided. The device can comprise a first reaction site and a second reaction site, fluidly connected to a combination site, wherein the combination site is fluidly connected to a first storage site; a third reaction site and a fourth reaction site, fluidly connected to a combination site, wherein the combination site is fluidly connected to a second storage site; a second combination site, fluidly connected to the first storage site and the second storage site; and an output, wherein the output allows a fluid in the second combination site to exit the device.

In some embodiments, a device for polymer synthesis is provided. The device can comprise a first reaction site and a second reaction site, fluidly connected to a purification site, followed by a combination site, wherein the combination site is fluidly connected to a storage site; a third reaction site and a fourth reaction site, fluidly connected to a purification site, followed by a combination site, wherein the combination site is fluidly connected to a second storage site; a second combination site, fluidly connected to the first storage site and the second storage site; an output, wherein the output allows a fluid in the second combination site to enter a final purification site and then exit the device.

### BRIEF DESCRIPTION OF THE DRAWINGS

FIG. 1A depicts a first part of various embodiments for serial/parallel polymer synthesis.

FIG. 1B depicts a second part of various embodiments for serial/parallel polymer synthesis.

FIG. 1C depicts an enlargement of the boxed section noted in FIG. 1A, depicting part of various embodiments for serial/parallel polymer synthesis.

### DETAILED DESCRIPTION OF EMBODIMENTS

Provided herein are devices and methods for the creation of polymers. In some embodiments, the devices and/or methods include numerous reaction sites, such as reaction wells or reaction sites, where short polymer fragments can be initially created as desired. In some embodiments, this occurs in parallel, for numerous reaction sites, thereby allowing numerous desired building fragments to be created, although each can be created in its own reaction site. The reaction sites can be connected via flow paths so as to allow the transportation of the fragments from at least two reaction sites into the same channel, chamber, or well for subsequent assembly. Thus, once a fragment (which can be as short as a single nucleotide or unit of the polymer) is created, the fragment can be released from the sites and then be orderly combined in a channel or a combination site or chamber. In some embodiments, this is repeated with numerous sites, thereby allowing the serial and/or parallel (and controlled) addition of fragments from numerous sites into a shared channel or channels, so as to lengthen the polymer in an ordered manner as it moves through the system. In some embodiments, the synthesis sites and channels are microfabricated, and can be made from things such as Si, glass, polymer based substrates, etc. In some embodiments, the synthesis of the polymer fragments in the sites is achieved by a first wavelength of light, while the release of the fragments from the sites are achieved via a second wavelength of light which can be the same or different from the first wavelength of light, where the first and second wavelengths of light do not unfavorably interfere with the other process (e.g., so there is no significant unintentional fragment release or creation at each site location). In such embodiments, the sites in the device are adequately transparent to the relevant wavelengths of light.

As the numerous reaction sites can be arranged in parallel and then flow together in pairs or in greater numbers, the combination of various desired building fragments can be controlled by the flow path arrangement and by the fragment release from the reaction sites. This allows for the desired building fragment to become extended and to become a first subpolymer (a larger desired building fragment). This subpolymer can then be stored in a “storage site,” while the device (or a subpart thereof) can be used or reused for the creation of a second or more desired building fragment and then a second of more subpolymer, each of which can be stored in separate storage sites. Once a sufficient number of subpolymers are created so as to include the sections of the desired full polymer, the subpolymers from the storage sites can then be combined together in a combination site to create the full length desired polymer. This combination of parallel synthesis, ordered combination, substorage, and then combination of the subpolymers allows for multiplexing reaction sites with much reduced size requirements for the synthesis of long polymers. In some embodiments, the polymers can be nucleic acids, such as DNA or RNA.

### DEFINITIONS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, “including,” “compose,” “composes,” and “composing” are not intended to be limiting. It is to be understood that both the foregoing general description and the following detailed description are exemplary and explanatory only and are not restrictive of the invention. The term “and/or” denotes that the provided possibilities can be used together or be used in the alternative. Thus, the term “and/or” denotes that both options exist for that set of possibilities.

Unless otherwise defined, scientific and technical terms used in connection with the invention described herein shall have the meanings that are commonly understood by those of ordinary skill in the art. Further, unless otherwise required by context, singular terms shall include pluralities and plural terms shall include the singular. Generally, nomenclatures utilized in connection with, and techniques of molecular biology, and protein and oligo- or polynucleotide chemistry and hybridization described herein are those well known and commonly used in the art. Standard techniques can be used, for example, for genetic material (nucleic acid) purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques of described herein are those well known and commonly used in the art.

The inventors are fully aware that they can be their own lexicographers if desired. The inventors expressly elect, as their own lexicographers, to use only the plain and ordinary meaning of terms in the specification and claims unless they clearly state otherwise.

As utilized in accordance with the embodiments provided herein, the following terms, unless otherwise indicated, shall be understood to have the following meanings:

The term “desired polymer” denotes the full length polymer that is to be created via the device and/or method disclosed herein. Other modifications can, of course, occur to the desired polymer on or after the use of the chip (including further monomer additions or hybridizations). In some embodiments, the desired polymer is at least 2 bases to 25,000 kbs in length on a single gene synthesis chip ranging from 1 reaction site to 10⁶ reaction sites. State of the art gene synthesis provides 96 to 1536 reaction sites permitting assembly of a polymer up to a maximum of 35 kbps.

The term “monomer” denotes an individual building block that makes up the polymer. Thus, for a nucleic acid, the monomer can be a nucleotide (natural or artificial).

The term “fragment” denotes a collection of two or more monomers, covalently linked to one another.

The term “desired building fragment” denotes a collection of monomers that have been linked together in a desired manner. The term “desired” denotes that it has a desired chemical composition. In the situation of nucleic acids, this can denote a desired nucleic acid sequence. In some embodiments, a “desired” sequence can be a unique and/or specified sequence, and in some embodiments, it can include a sequence that can be used for subsequent hybridization events in assembling the first-, second-, third- fourth-, level etc. fragments. In some embodiments, the desired building fragment comprises, consists, or consists essentially of random monomers, such as random nucleotides. In some embodiments, the desired building fragment does not include random nucleotides.

The term “first-level fragment” denotes a fragment that comprises at least two desired building fragments. The term “second-level fragment” denotes a fragment that comprises at least two first-level fragments. The term “third-level fragment” denotes a fragment that comprises at least two second-level fragments. The term “fourth-level fragment” denotes a fragment that comprises at least two third-level fragments. The term “fifth-level fragment” denotes a fragment that comprises at least two fourth-level fragments. In theory, there is no limit to the number of possible levels of fragments that one can have. Thus, the “xxxx-level fragment” can be so designated without limitation (thus, sixth, seventh, eight, to n level fragments are described herein).

A “ligated” fragment indicates that the fragment has been ligated together so as to remove any single stranded breaks in the fragment. Thus, the individual strands are now adequately continuous. In the situation of nucleic acids, this denotes that the individual strands of the double stranded fragment have been ligated together (e.g., which can be done via ligase or by chemical approaches) such that there are two continuous strands, hybridized to one another. When used with non nucleic acid embodiments, “ligated” simply denotes that the fragments have been linked together.

The term “subpolymer” denotes a collection of two or more fragments, covalently linked to one another which are then stored in a storage site.

The term “oligo” denotes an oligonucleotide which is a single-stranded nucleic acid that can be DNA, RNA, PNA and/or other nucleic acid analog that is typically 2 to 500 monomers in length.

The term “reaction site” denotes an area (such as a chamber or well), where a specific chemical reaction can occur in relative isolation from other such reaction sites. In some embodiments, the reaction sites allow for the transmission of various wavelengths of light, which can, in some embodiments, induce fragment production from the monomers and/or release the fragment from a wall or surface of a reaction site.

The term “storage site” denotes a location that is isolated from another storage site. A subpolymer or fragment can be stored in a storage site while the upstream system can be reused to produce a separate subpolymer.

The term “overlap” when referring to the absence of any overlap between a wavelength of light for coupling and a wavelength of light for separation (or detachment) denotes that any extent of optical overlap is not problematic for the system being used. As will be appreciated, various chemical entities may absorb a range of wavelengths, rather than simply one or two wavelengths; however, the efficiency of this absorption spectra at the extremes can be low enough that there will not be any problematic results from the overlap (e.g., any amount of inadvertent coupling and/or separations will be within noise levels or an insignificant amount of the product). In some embodiments, there is absolutely no overlap. In some embodiments, any overlap results in at least a 100 fold bias to one of the events over the other event (for example, at least 1000, 10,000, 100,000, 1,000,000 or more bias). In some embodiments, the wavelength of coupling and the wavelength for separation (or detachment) of oligonucleotides is the same wavelength or overlap. In some embodiments, this can be achieved by use of a photogenerated cleavage reagent.

The term “selectively irradiate” denotes that light energy (of any wavelength) is being directed to a desired location. In some embodiments, light is selectively directed to a first location while not being directed to a second location (such as a second reaction site). In some embodiments, some light can “bleed” onto surrounding reaction sites, however, the intensity of light will not be large enough so as to create significant amounts of unintended reactions (coupling and/or separations (or detachments)). Light can be directed in any number of ways, including, mirrors, filters, lens, etc.

The term “effectively transparent” denotes that a structure allows a sufficient amount of light of a desired wavelength to pass through the structure. As will be appreciated by one of skill in the art, some light will and can be absorbed by the structure, as long as an amount that is sufficient to serve its purpose makes it to the desired location (such as within the reaction site). The degree of transparency can also vary based upon the strength of the light used in the system.

The term “combination site” denotes a location, which can be a chamber or well, where two or more previously separate channels or flow paths are combined into a common volume. The combination site can include a flow path itself, as well as a chamber that the flow path empties into. In some embodiments, the combination site is positioned next to a heating and/or cooling element so as to allow the heating and/or cooling of the sample in the combination site.

As used herein, “polynucleotide” is intended to mean two or more nucleotides linked together through a covalent bond. For example, nucleotides can be linked together through a phosphodiester bond. A polynucleotide can contain the four nucleotides adenine, guanine, cytosine, and thymine or nucleotide analogues and derivatives such as inosine, dideoxynucleotides or thiol derivatives of nucleotides. Different chemical forms of nucleotides such as nucleosides or phosphoramidites can be used to generate a polynucleotide. In addition, nucleotides can further incorporate a detectable moiety such as a radiolabel, a fluorochrome, a ferromagnetic substance, a luminescent tag or a detectable moiety such as biotin Strepavidin, a chemical linker for subsequent synthesis and/or addition of branching polymer and small molecule side-chains. Polynucleotides also include, for example, RNA, peptide nucleic acids (PNAs), locked nucleic acids (LNAs) and other polynucleotide variations commonly know in the art and/or combinations of said polynucleotides.

### Detailed Embodiments

Herein provided are various embodiments for preparing polymers. In some embodiments, the polymers synthesized are DNA, RNA, proteins, or oligosaccharides, via numerous (e.g., hundreds, thousands or more, e.g., 7776) reaction sites where fragments (e.g., pieces of DNA or RNA) can be orderly assembled.

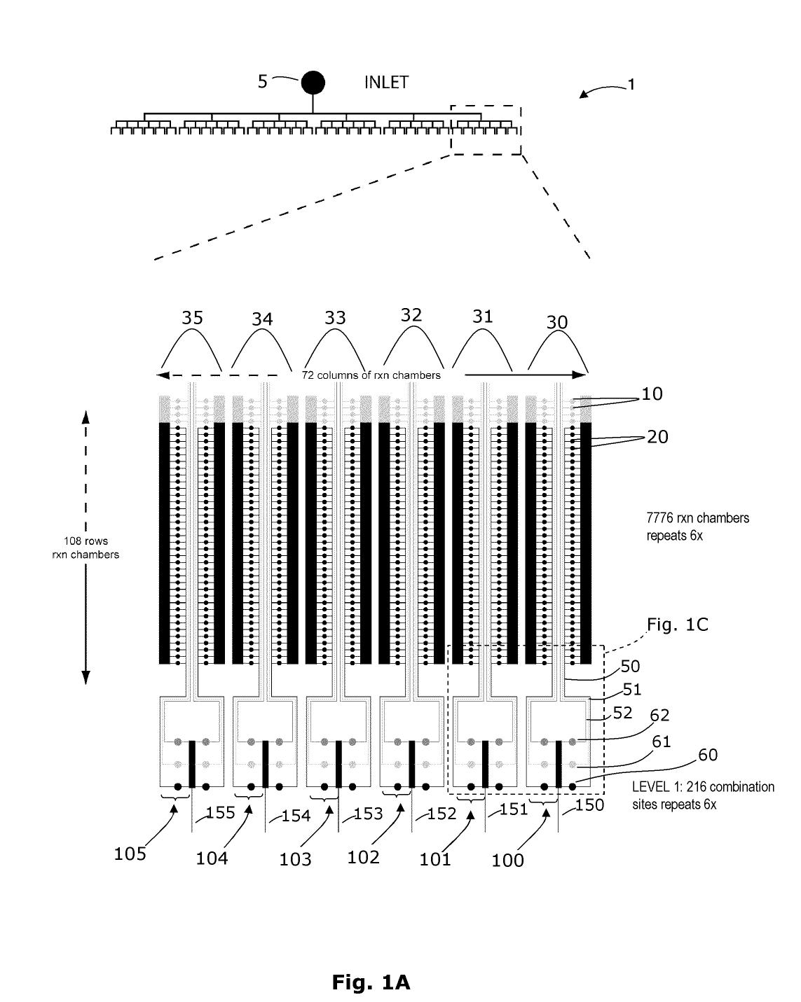

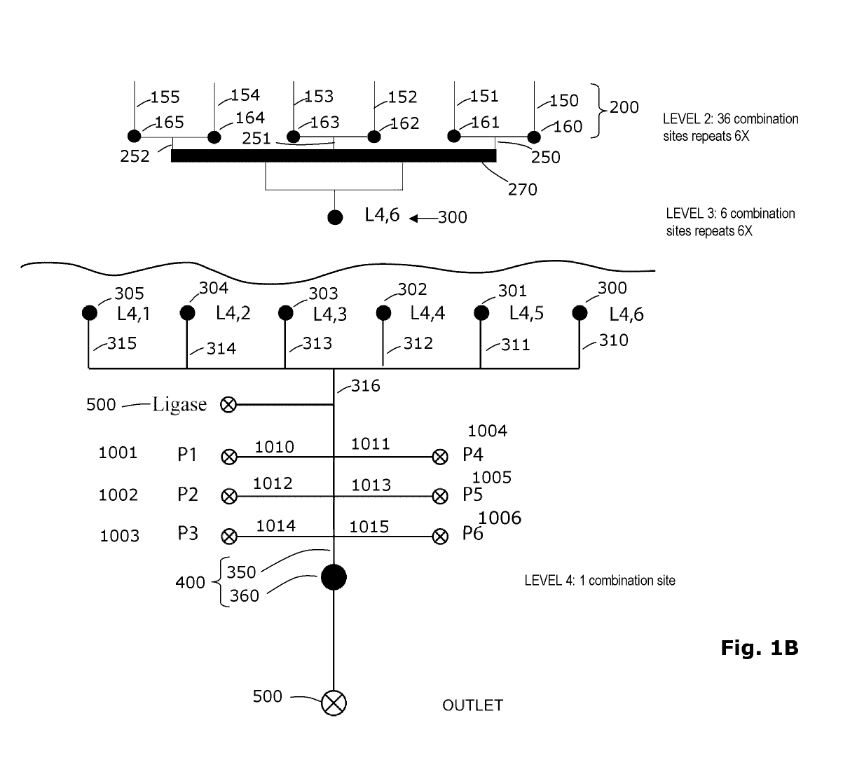

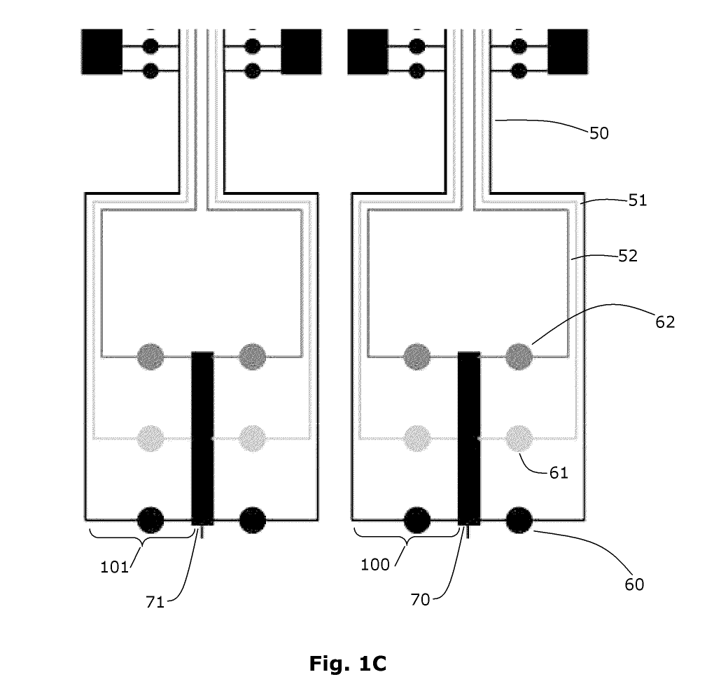

Some embodiments of a device for preparing polymers are shown in FIGS. 1A-1C. A blow up of the device 1 is shown in the bottom half of FIG. 1A and continuing to FIG. 1B. As shown, in some embodiments, numerous reaction sites 10 and 20 are provided on a surface. The reaction sites are fluidly linked together in sets, such that numerous reaction sites are linked via a flow path (50 for reaction sites 20 and 51 for reaction sites 10) to a combination chamber 60 (61 and 62), which can be used to mix the products (desired building fragments) together to form a first-level fragment. Other reaction sites (not shown) can also be linked into the system via additional flow path 52, which lead into a larger flow path 70, which in turn lead to a second level combination site and a second level combination chamber 160. As will be appreciated by those in the art, products from the various reaction sites can be combined, both in the flow paths 50, 51, and 52, as the products leave the reaction sites 10, 20, etc., as well as in the chamber 60 itself. This combination of areas (e.g., 50, 51, 52, and 60) is generally denoted as the combination site 100. Within the combination chamber 60, when nucleic acids are being assembled, the combination site can be controlled so as to alter the temperature of the internal solution so as to allow for selective/specific hybridization events and ligation events. The combination sites 100, 101, 102, 103, 104, and 105 at this point are denoted as “first-level combination sites”.

Following the formation of the first-level fragment(s), a combination reaction or linking reaction (e.g., ligation reaction) can be performed in the device in some embodiments to covalently connect the various parts of the first-level fragment; thus, in some embodiments, this section of the device is configured for the ability to alter solution parameters for ligation reactions (e.g., temperature, buffers, ligase, etc.)

The first-level combination sites are connected, optionally via flow paths 70, 71, etc and then optionally via further flow paths 150, 151, 152, 153, 154, and 155 to second-level combination chambers 160, 161, 162, 163, 164, and 165 (FIG. 1B), where the second-level combination site 200 comprises both the second-level combination chambers 160, 161, 162, 163, 164, and 165 and the flow paths (150, 151, 152, 153, 154, and 155), as well as the larger flow paths 70, 71, etc. As above, this second-level combination site 200 and/or chamber 160, 161, 162, 163, 164, and 165 can be configured for the addition of various ingredients into the system and can be configured for heating and cooling.

This “collapsing” process can be repeated any number of times (1, 2, 3, 4, 5, 6, 7, 8, 9, 10, 100, 1000, etc or more times) to allow further combinations; however, as depicted in FIG. 1B, eventually the collapsing structure is stopped and linked to a storage site 1001 via one or more flow paths 310 and 316. The storage site can be reversibly fluidly sealed from the above noted upstream section of the device, so that a first subpolymer produced via the above process can be stored in the storage site, while the device is used to create one or more additional subpolymers, each of which can be placed into a separate storage site 1002, 1003, 1004, 1005, and 1006. There is no limit to the number of storage sites employed and in some embodiments 1, 2, 3, 4, 5, 6, 7, 8, 9, 10, 11, 12, 13, 14, 15, 20, 25, 30, 35, 40, 45, 50, 60, 70, 80, 90, 100, 200, 300, 400, 500, 600, 700, 800, 900, 1000 or more storage sites are employed or on a device.

The storage sites (1001, 1002, 1003, 1004, 1005, and 1006) are linked together via a flow path 1010, 1011, 1012, 1013, 1014, and 1015 and into a fourth-level combination site 400, which can include a flow chamber 350 and a combination chamber 360. If needed a further source of ligase 500 (or other linking agent) can be provided to provide for additional ligase for the combination site. As noted above, the combination site 400 can include or be proximal to a heating element so as to allow the manipulation of the temperature of the solution. Finally, an outlet 500 can be provided, by which a produced polymer can be collected. In some embodiments, the device includes one or more optional valves (indicated in FIG. 1B via a circled “X”). In some embodiments, the one or more valves are located off of the device, and thus, the valve need not be part of the chip itself.

As will be appreciated, the above arrangements can be altered in varying ways. Furthermore, various embodiments for each of the above noted parts are provided below.

In some embodiments, the number of levels (for each level of combination chamber and/or combination site) can be increased or decreased. In some embodiments, there is 1, 2, 3, 4, 5, 6, 7, 8, 9, 10 or more levels where two or more previous sites (which can be reaction sites and/or combination sites and/or storage sites) are collapsed to a single or fewer sites. In some embodiments, there are more than 10 levels,

At each of the sites, and at the start, end or anywhere in-between for the flow paths, there can be a valve to control flow in or out of the site as well as a liquid inlet, allowing one to add liquid to the system. The valves can be selected from the following group: solenoid, ball, diaphragm, piston, needle, magnetic, pinch, thermal expansion/contraction, memory polymers, check valves or any other valve device capable of handling the reagents and pressures required for operation.

In some embodiments, the combination sites comprise a flow path and a combination chamber. In some embodiments, the flow paths converge and no distinct combination chamber is required (although a heating element can still be associated with each of these structures).

In some embodiments the manifold and valves for controlling the fluid inputs can also be manufactured into the polymer synthesis chip. In some embodiments, the inlet has a switching valve to allow for different reactants to be added. In some embodiments, the represented chip has 108 rows of reaction sites (not all shown) by 72 columns (not all shown) of reaction sites for a total of 7776 reaction sites. In the columns the 108 rows of reaction sites are collapsed 36 at a time into a single channel represented in FIG. 1A by the solid, light dotted, or dark dotted lines. FIG. 1A only shows the lower 40 reaction sites (10, 20), 36 of which are black 20 with the remaining 4 being grey 10. Although a 108×72 array is depicted in FIG. 1A any array size is possible given the limitations in manufacturing. In some embodiments, the device comprises a Si wafer. In some embodiments, the Si wafers are in the range for 25 mm to 300 mm, therefore array sizes up to 100000 by 100000 are possible on a single wafer. In some embodiments, the Si wafers are 35 nm to 300 mm. In some embodiments, the Si wafers are 35 nm to 3 m.

### Nucleic Acid Embodiments

Some embodiments of FIGS. 1A-1C include a device and method for manufacturing nucleic acids. Using FIG. 1A as an example, an inlet 5 can be used to add A, C, G, T, U or any modified nucleic acid or nucleic acid analogue precursors, as well as a buffer solution and ligase to the device as a synthesis procedure demands. This inlet channel 5 splits to feed all columns 30, 31, 32, 33, 34, and 35 of the reaction site array portion. In some embodiments, there can be more than one inlet to feed the synthesis reagents. In some embodiments, there are at least 8 other ports on the chip: at least one port for the perfusion of additional ligase to supplement that depleted throughout the process, at least six ports to store and dispense segments of DNA and an outlet 500 through which waste and the final product are dispensed. In some embodiments, the ports, inlets, and outlets are equipped with on/off valves located off the DNA synthesis chip.

In some embodiments, linkage of nucleic acid monomers in the reaction site is achieved through the use of a photogenerated reagent, such as a photogenerated acid, base, enzyme, etc. through individually addressable digital light processing (DLP) projection technology allowing individual reaction sites (10, 20 shown in FIG. 1A) to be addressed like pixels on a screen. Through this process a piece of single stranded DNA of any sequence and incremental length can be obtained.

In some embodiments, cleavage of the ssDNA (which is the desired building fragment in these embodiments) from walls of the reaction sites can be achieved through the use of a photogenerated reagent, such as a photogenerated acid, base, enzyme, etc. through individually addressable digital light processing (DLP) projection technology allowing individual reaction sites (10, 20 shown in FIG. 1A) to be addressed like pixels on a screen. Cleavage of the ssDNA (which is the desired building fragment in these embodiments) from the walls of the reaction sites can also be achieved by a photodegradable linker molecule anchoring the ssDNA to the surface of the reaction sites. In such embodiments, the wavelength of light used in synthesis will not meaningfully interfere with the wavelength of light used in cleavage. The same wavelength may be used for coupling and cleavage when the photogenerated reagent needed for coupling is made in a light addressed pixel only in the presence of the correct photoactivatable coupling precursor and the photogenerated reagent needed for cleavage is made in a light addressed pixel only in the presence of the correct photoactivatable cleavage precursor. In some embodiments, this combination of photoacid generation followed by an indexable photocleavage allows DNA synthesis to occur without the use of on chip valves. In some embodiments NH₄OH or methylamine can be used. In some embodiments valves are used to gate the contents of the reaction sites into the combination sites. In this case, the ssDNA is lyophilized before assembling strands of dsDNA to remove the NH₄OH. In some embodiments, situation deprotection is achieved using gas phase ammonium hydroxide, methalymine or other gas phase deprotection agent.

Following the production of the desired building fragment, a purification process can be performed between the desired building fragment production and the combination of two or more desired building fragments into a first-level fragment. In some embodiments, the purification is a size exclusion purification that removes or reduces the number of relatively short single stranded fragments from entering the combination chamber 60 or the combination site 100. In some embodiments there is a two phase purification. Purification No. 1 can be a purification of oligos that are still attached to the reaction site surface. Purification No. 2. can be a purification of the sample once the product has been detached from the surface of the reaction site.

The present invention also provides improved methods for deprotecting, washing, and releasing polymers synthesized on the derivatized substrate. In particular, as described above, the anchor moiety is selectively photocleavable. As the growing polymer chain is continually deprotected under basic conditions, such as EDA in anhydrous EtOH, the polymer attached to the substrate is not cleaved from the substrate. Upon completion of the deprotection reactions, the substrate surface is rinsed to remove small molecular fragments resulting from the deprotection. This provides a surface with the polymer attached that is free of salt and other small molecular contaminants. The polymers are then removed from the substrate through photo-initiated cleavage reaction. Purification No. 1 which is purification of oligos that are still attached to the reaction site surface is taught in US 2003/0120035 A1, the entirety of which is herein incorporated by reference.

Following the production of the desired building fragment, a ligase suspended in a buffer solution can then be perfused through the reaction sites which collapse together (e.g., 36 at a time into 216 combination chambers 60, 61, and 62 via flow paths 50, 51, and 52, where ssDNA (the desired building fragment in this embodiment) is assembled into double stranded DNA (dsDNA the first-level fragment in this embodiment). These in turn are combined (e.g., 6 at a time) into a larger flow path 70 (71, etc.). This method of multiplexing 2 or more of the desired building fragments (e.g., ssDNA) produced from the reaction sites into a single flow path 50 or combination chamber 60 allows for a conservative footprint for the chip. In some embodiments, reaction sites 10 and 20 feed independent flow paths 50, 51, and 52 before being combined in the combination sites. These fluidic channels connecting reaction sites to combination sites 100, 101, 102, 103, 104, 105 (or combination chambers 60, 61, 62) can be in the range of 1 mm (radius of dsDNA) to 10 mm, for example 10 nm to 1 mm or 100 nm to 100 micrometers. In some embodiments, the combination chambers 60, 61, and 62 are optional and can be omitted; thus, combination of the fragments occur within the flow paths and as the various flow paths merge together.

Once enough fluid has been added to displace the cleaved ssDNA (desired building fragment in this embodiment) into the combination sites the chip (or the subpart thereof that is proximal to the combination site) undergoes a temperature cycle to anneal and ligate the DNA pieces together.

In some embodiments, at level 1 combination assemblies, 6 pieces of ssDNA combine to form a single piece of dsDNA three linear strands or increments long (i.e., three forward and three reverse strands or increments) with overlap of complementary base sequences. The subsequent assembly levels then bring pieces of dsDNA together for annealing and ligation forming pieces of dsDNA 18, 108 and 648 linear increments long. After the dsDNA reaches 648 DNA linear increments in length, given a 50 base long ssDNA increment, the length of the dsDNA is approximately 32,400 base pairs long. This length of dsDNA is on the order of most viral genomes ranging from 10 kbps to 50 kbps. This process of combining, annealing; and ligating is continued through to the storage sites.

After the DNA contents have surpassed level 3 and/or flow path 316 it is stored in 1 of 6 channels or sites hereto after referred to as storage sites 1001, 1002, 1003, 1004, 1005, and 1006. This area of the chip can be isolated from the temperature cycling from the previous levels 1 through 3 or 4 and held at less than 310 K until storage sites 1 to 6 are as full as desired (or the appropriate number of storage sites is as full as desired).

The storage sites function to allow the fabrication, collapse and assembly of a large number of ssDNA segments on a reasonably sized single chip with a single or multiple levels from a large number of reaction sites. In some embodiments, the DNA pieces are driven into these storage sites through gas and/or liquid and/or osmotic and/or capillary and/or chemical potential differential via a simple off chip valving system. In some embodiments, the same length strand of DNA can be produced using a number of chips equal to the storage sites in the aforementioned embodiment and combined on a subsequent chip or combined later through common practices. By opening the valve controlling the port of DNA storage sites 1, fluid can be perfused though the open port pushing the DNA into 1001 (P1, storage sites 1) while the ports controlling 1002-1006 (P2 through P6) and the outlet are closed. For the next assembly run P1 (1001) is closed and the outlet is opened and the process repeats however on this pass after the DNA reaches the level above the storage sites, instead P2 1002 is opened while P1 1001, 1003-1006 (P3 through P6) and the outlet are closed.

Once all (or the desired number of) the storage sites are full, buffer is perfused through the ports on P1 though P6 and ligase is added through the port directly upstream of the storage site pushing the contents of P1 through P6 into the final combination site 400 to be ligated. The result is a dsDNA assembly, given a 50 base long ssDNA increment, of 194 kbps (the desired polymer in this embodiment). In some embodiments, this is the size of a minimal genome operating system in one on chip synthesis.

In some embodiments, this process method is can be employed for the simple low cost fabrication of such a device in a single layer without the need for complex components such as valves or multi layer bonded chip and impacts the manufacturability and cost of such a chip. In some embodiments, if storage sites were not designed into the final combination level the number of channels leaving the first level would make such a device unmanufacturable due to the large number of individually indexable valves required and/or the size. For example, if 7776 channels were individually plumbed out of the first level given a 1 micron channel and 1 micron step 7776 channels would occupy a length of 15 mm however the pressure drop to be overcome would be substantial. Even if the dimension of the channel is only increased to 10 microns the chip then becomes approximately 77 mm, too large to obtain a substantial number of the devices on any single Si wafer (given a common Si wafer diameter range of 25 mm to 300 mm).

### DNA Fragment Synthesis

As will be appreciated by those in the art, there are a number of ways of producing nucleic acids.

Methods for synthesizing polynucleotides (also known as oligonucleotides) are known in the art and can be found described in, for example, Oligonucleotide Synthesis: A Practical Approach, Gate, ed., IRL Press, Oxford (1984); Weiler et al., Anal. Biochem. 243: 218 (1996); Maskos et al., Nucleic Acids Res. 20 (7): 1679 (1992); Atkinson et al., Solid-Phase Synthesis of Oligodeoxyribonucleotides by the Phosphitetriester Method, in Oligonucleotide Synthesis 35 (M. J. Gait ed., 1984); Blackburn and Gait (eds.), Nucleic Acids in Chemistry and Biology, Second Edition, New York: Oxford University Press (1996), and in Ansubel et al., Current Protocols in Molecular Biology, John Wiley and Sons, Baltimore, Md. (1999). All of the above references are incorporated herein by reference in their entirety.

Solid-phase synthesis methods for generating arrays of polynucleotides and other polymer sequences can be found described in, for example, Pirrung et al., U.S. Pat. No. 5,143,854 (see also PCT Application No. WO 90/15070), Fodor et al., PCT Application No. WO 92/10092; Fodor et al., Science (1991) 251: 767-777, and Winkler et al., U.S. Pat. No. 6,136,269; Southern et al. PCT Application No. WO 89/10977, and Blanchard PCT Application No. WO 98/41531. Such methods include synthesis and printing of arrays using micropins, photolithography and ink jet synthesis of oligonucleotide arrays. Methods for synthesizing large nucleic acid polymers by sequential annealing of polynucleotides can be found described in, for example, in PCT application No. WO 99/14318 to Evans and U.S. Pat. No. 6,521,427 to Evans. All of the above references are incorporated herein by reference in their entirety.

Polynucleotides can be generated on commercial nucleic acid synthesizers using phosphoramidite chemistry. The Practical Approach series has reviewed phosphoramidite and alternative synthetic strategies (Brown, T., and Dorkas, J. S. Oligonucleotides and Analogues a Practical approach, Ed. F. Eckstien, IRL Press Oxford UK (1995)). Chemical synthesis of polynucleotides is a process in which four building blocks (base phosphoramidites) are connected as a linear polymer. In addition to the component bases, a number of reagents are required to assist in the formation of internucleotide bonds, oxidize, cap, detritylate, and deprotect. Automated synthesis can be performed on a solid support matrix that serves as a scaffold for the sequential chemical reactions; a series of valves and timers to deliver the reagents to the matrix, and finally a post-synthesis processing stream that can include purification, quantification, product QC, and lyophilization. Some of the standard DNA bases (G, C, and A) contain primary amines that are reactive; therefore, the primary exocyclic amines can be modified with protecting groups so as to not participate in unwanted reactions during synthesis. Further, the four phosphoramidites contain a phosphorus linkage that similarly needs to be protected. Chemical groups used to protect these sensitive sites can remain intact during the DNA synthesis cycle yet can be readily removed after synthesis so that normal, unmodified DNA results. A number of different protecting strategies have been developed. For example, phosphoramidites with b-cyanoethyl protected phosphorus can be used. For the heterocyclic bases, protection of primary amines is often provided by a benzyol group for adenine and cytosine and either a dimethylfonrnamidine or isobutyrl group for guanine. Thymine, which lacks a primary amine, does not require base protection. These protecting groups are stable under conditions used during synthesis, but are rapidly and effectively removed by treatment with ammonia. It is also desirable to block the 5′-OH of the base-phosphoramidites so that activated monomers do not react with themselves but can only react with the 5′-OH on the growing polynucleotide chain tethered to the solid support. Current chemistry, for example, employs a dimethoxytrityl (DMT) group. After condensation, the DMT group is cleaved from the newly added DNA base by treatment with acid. Released DMT cation is orange and progress of the DNA coupling efficiency can be monitored by spectrophotometric reading at 490 nm.

The 3′ hydroxyl group of the deoxyribose sugar is derivatized with a highly reactive phosphitylating agent. The phosphate oxygen on this group is usually masked by the 13-cyanoethyl moiety that can be removed by 13-elimination using ammonia hydroxide treatment at elevated temperatures.

Automated synthesis can be done on solid supports. In some embodiments, bases are added to the growing chain in a 3′ to 5′ direction (opposite to enzymatic synthesis by DNA polymerases).

Although “universal” supports exist, synthesis is more often begun using a surface that is already derivatized with the first base, which is attached via an ester linkage at the 3′-hydroxyl.

Synthesis starts with the first base (which can be a monomer, dimer, trimer, tetramer, pentamer, or hexamer nucleotide phosphoramidite) attached to the solid support and elongates in a 3′ to 5′ direction.

The polynucleotide synthesis cycle can proceed in four steps as described below: (1) De-blocking; (2) Activation/coupling; (3) Capping; and (4) Oxidation.

Deblocking: The synthesis cycle begins with the removal of the DMT group from the 5′ hydroxyl of the 5′-terminal base by brief exposure to dichloroacetic acid (DCA) or trichloroacefic acid (TCA) in dichloromethane (DCM). The yield of the resulting trityl cation can be measured to help monitor the efficiency of the synthetic reaction. Protection of the reactive species (primary amines and free hydroxyls) on the nucleoside building blocks insures that the exposed 5′-hydroxyl is the only reactive nucleophile capable of participating in the coupling reaction (next step).

Activation/Coupling: DNA (monomer, dimer, trimer, tetramer, pentamer, hexamer nucleotide) phosphoramidites are converted to a more reactive form by treatment in tetrazole or a tetrazole derivative at the time of coupling. These processes occur through the rapid deprotonation of the phosphoramidite followed by the reversible and relatively slow formation of a phosphorotetrazolide intermediate. Coupling reactions with activated deoxyribonucleoside-phosphoramidite reagents are fast and efficient.

An, excess of tetrazole over the phosphoramidite can be used to ensure complete activation and an excess of phosphoramidite over reactive polynucleotide coupled to CPG. Under these types of conditions coupling efficiencies of >99% can be achieved.

Capping: Since the base-coupling reaction is not 100% efficient, a small percentage of the growing polynucleotides on CPG supports will fail to couple and result in undesired, truncated species. Unless blocked, these truncated or partial polynucleotide products can continue to function as a substrate in later cycles, extend, and result in near full-length polynucleotides with internal deletions. These truncated or partial polynucleotide products are called (n−1)-mer species. These “reaction failures” can be mostly prevented from participating in subsequent synthesis cycles by “capping”, which involves acetylation of the free 5′-OH with acetic anhydride.

Oxidation: At this point, the DNA bases are connected by a potentially unstable trivalent phosphite triester. This species is converted to the stable pentavalent phosphotriester linkage by oxidation. Treatment of the reaction product with dilute iodine in water/pyridine/tetrahydrofuran forms an iodine-phosphorous adduct that is hydrolyzed to yield pentavalent phosphorous. The oxidation step completes one cycle of polynucleotide synthesis; subsequent cycles begin with the removal of the 5′-DMT from the newly added 5′-base.

Cleavage and Deprotection: After synthesis is complete, in some embodiments, the polynucleotide can be cleaved from the solid support with concentrated ammonium hydroxide at room temperature. Continued incubation in ammonia at elevated temperature will deprotect the phosphorus via B-elimination of the cyanoethyl group and also removes the protecting groups from the heterocyclic bases. In some embodiments, as noted above, the cleavage can occur via a photochemical reaction.

Post Synthesis Handling: During synthesis, both full-length polynucleotides and truncation products or partial polynucleotide products remain attached to the CPG support. Following synthesis, in some embodiments, the species are similarly cleaved and recovered so that the final reaction product is a heterogeneous mixture of wanted and unwanted species. Impurities accumulate to a greater degree as polynucleotide length increases. Furthermore, cleaved protecting groups are also present. At this point, polynucleotides are traditionally “desalted”, a process in which small molecule impurities (protecting groups and short truncation products) are removed using gel filtration or organic solid-phase extraction (SPE) methods.

Use of desalted polynucleotides with no additional purification can be appropriate when using short primers in simple applications, such as routine PCR or DNA sequencing. However, n−1 and other truncation or partial polynucleotide species can lead to deletion mutants if used in cloning, site-directed mutagenesis or gene assembly applications.

In some embodiments, purification by PAGE or HPLC can be used to remove truncated or partial polynucleotide species. In some embodiments, the polymer length is kept short to reduce errors. In other embodiments a filter step is used to exclude fragments under the desired fragment size. Of course, other steps are also possible for filtering the undesired partial fragments from the full length fragments.

Polynucleotide synthesis efficiency is typically about 98-99% for each cycle of chemistry, so for each cycle about 1-2% of the reaction products will be 1 base shorter than expected. Some truncated species fail “capping” and continue to participate in additional cycles of DNA synthesis. For a 60-mer polynucleotide, less than 50% of the final product will be the desired full-length molecules. The final synthesis product will include a mixed population of (n−1)-mer and (n−2)-mer (etc.) molecules which represent a heterogeneous collection of sequences, effectively a pool of deletion mutants at every possible position.

Synthesis scale refers to the amount of starting material while synthesis yield refers to the amount of final product recovered after the synthesis and purification steps have been completed. In polynucleotide synthesis, the 3′ terminal base is attached to a solid support at the scale ordered by the customer. Bases are added one at a time in the 3′ to 5′ direction. Ideally, each added base would couple with 100% efficiency, resulting in 100% yields. In reality, coupling efficiency is somewhat less than 100%, and this small decrease can result in a substantial decrease in yield of the final oligonucleotide (since the effects of coupling efficiency will be additive). Moreover, coupling efficiency can vary for each base added, therefore the sequence itself can contribute to wide variations in yields. For a 250-nmole-scale reaction, the final yield after deprotection and purification can range from 10 to 100 nmoles. Some sequences tend to produce higher yields than others, and this trend is usually reproducible. The yield for the synthesis of one 20-base sequence can be twice that obtained for a different 20-base sequence, even if the two sequences are run on the same day, on the same machine, using the same reagents. Some variability in yields can also be derived from the individual machine used.

Theoretical yield for a given synthesis is (Eff) n−1 with Eff representing coupling efficiency and ‘n’ representing the number of bases in the polynucleotide. If the coupling efficiency is 99% (Eff=0.99), the fraction of full-length product present after synthesis will be approximately (0.99)¹⁹ or 83% for a 20-mer; (0.99)⁴⁹ or 61% for a 50-mer; and (0.99)⁷⁴ or 48% for a 75-mer. A small decrease in coupling efficiency will result in a substantial decrease in expected yield. For example, if coupling efficiency is 99%, the yield for a 100-mer is (0.99)⁹⁹ or 37%, but if the coupling efficiency drops to 98%, yield falls to (0.98)⁹⁹ or 13%.

However, coupling efficiency varies with each base added. Coupling efficiency is lower for the first five to six bases, presumably because of steric hindrance near the surface of the solid support. Coupling efficiency then increases to an optimum of about 99%, as is characteristic for the addition of the twentieth base, and then once again, falls to suboptimal levels as length increases. Since coupling efficiency actually decreases as the polynucleotide becomes very long, yields on 100-mets can often be less than 10%. Product is also lost during any purification process, if done, which further decreases yields.

In some embodiments, each polynucleotide produced can be checked for quality or a sampling of the polynucleotides produced can be selected and tested for quality, for example, on a Beckman P/ACE MDQ 96-well CE, and/or HPLC, and/or mass spectrometer. The purity is calculated by taking the percent area of the polynucleotide main peak, or N of the CE or HPLC curve.

### Photo Generated Reagents—Including Nucleic Acid Synthesis

In some embodiments, instead of adding monomers and reagents serially to a reaction site in order to produce a longer fragment, one can make or activate the various monomers for creating the fragments by using photo generated reagents.

In some embodiments, one can use the methods and compositions outlined in U.S. Pat. No. 6,426,184 to attach an initial monomer to a surface of a reaction site (herein incorporated by reference in its entirety and in regard to this aspect).

In some embodiments, one can use the methods and compositions outlined in U.S. Pat. Pub. 20030143131 for selectively converting photogenerated precursors to photogenerated reagents, thereby allowing for further fragment formation (herein incorporated by reference in its entirety and in regard to this aspect).

In some embodiments, one can use the methods, devices, and compositions outlined in U.S. Pat. Pubs. 2003/0138363, 2003/0186427, 2004/0023368, and PCT Pub. WO 99/41007 for deprotecting and initiating moiety attachment to a wall for further fragment synthesis (herein incorporated by reference in their entireties and in regard to this aspect).

In some embodiments, one can use the methods and compositions outlined in U.S. Pat. Pub. 2003/0120035 in regard to selectively cleavable linkers (herein incorporated by reference in its entirety and in regard to this aspect). Such linkers can be included or used to attach the first monomer (or subsequent monomer) to the surface of the reaction site such that the desired building fragment can be optically cleaved from the surface at a later point in time.

In some embodiments, the compositions and methods for separating and/or detaching the fragments from the reaction site wall involves one or more of the embodiments in U.S. Pat. No. 6,586,211 (herein incorporated by reference in its entirety and in regard to this aspect).

In some embodiments, the desired building fragments used for synthesizing the polymer are 5-250, 10-200, 10-100, or 10-50 monomers in length. In successive steps, it is possible to detach in each case partially complementary desired building fragments front the reaction site and to bring them into contact with one another under hybridization conditions in a combination site.

In some embodiments, when the polymer is a nucleic acid, the base sequences of the desired building fragments synthesized in individual reaction sites are chosen such that they can assemble to form a nucleic acid double strand hybrid (which will be the first-level fragment in this embodiment). The desired building fragments can then be separated from the wall in one or more steps under conditions such that a plurality, i.e. at least some of the separated desired building fragments assemble to form a first-level fragment. Subsequently, the nucleic acid fragments forming one strand of the first-level fragment can at least partially be linked covalently to one another. This can be carried out by enzymatic treatment, for example using ligase, or/and filling in gaps in the strands using. DNA polymerase.

In some embodiments, the desired single strand fragments are synthesized in a multiplicity of reaction areas on a reaction support by in situ synthesis. This can take place, for example, using the supports described in the patent applications DE 199 24 327.1, DE 199 40 749.5, PCT/EP99/06316 and PCT/EP99/06317. In this connection, each reaction area is suitable for the individual and specific synthesis of an individual given DNA sequence of approx. 10-250 nucleotides in length. These DNA strands form the building blocks for the specific synthesis of very long DNA molecules.

In some embodiments, the reaction site synthesis is carried out by light-dependent location- or/and time-resolved DNA synthesis in a fluidic microprocessor which is also described in the patent applications DE 199 24 327.1, DE 199 40 749.5, PCT/EP99/06316 and PCT/EP99/06317 (herein incorporated by reference in their entireties).

### Fragment Separation or Detachment

There are a number of ways to break a bond or an association between a desired building fragment and a solid surface (such as the surface of a reaction site). In some embodiments, this is done via photocleavage and thus a photo cleavable moiety is employed to connect the fragment to the substrate of or within the reaction site. For example, photocleavable moieties may consist of pivaloyl linkers, phenacyl esters, o-nitrobenzyl photocleavable linker, dimethoxynitrobenzyl moiety. In other embodiments the photocleavable moiety is 8-bromo-7-hydroxyquinoline. In other embodiments the photocleavable moiety is nitrodibenzofuran. In other embodiments the photocleavable moiety is 6-bromo-7-hydroxycoumarin-4-ylmethyl. The linkers find use in synthetic methods, including the generation of photocleavable oligonucleotides, e.g. caged morpholinos. U.S. Patent Application 20100022761 (herein incorporated by reference in its entirety and in regard to this aspect).

In some embodiments, the cleavage is done by random access photocleavage (RAP) (or random access cleavage “RAC”).

In some embodiments, fragment separation is achieved by chemical cleavage.

In some embodiments, the separation of the desired building fragment is achieved via heat based cleavage. In some embodiments, the separation of the desired building fragment is achieved by a change in the acoustic, magnetic and/or electrical field aspects of the walls of the reaction site. In some embodiments, the separation or detachment of the desired building fragment is achieved by a change in chemisorption- and/or physisorbtion aspects of the walls of the reaction site. In some embodiments, the separation or detachment of the desired building fragment is achieved by photogenerated reagent cleavage of appropriate linker. In some embodiments, the separation or detachment of the desired building fragment is achieved by a change in receptor-substrate binding aspects of the walls of the reaction site. In some embodiments, the separation or detachment of the desired building fragment is achieved by a change in chelation aspects of the walls of the reaction site. In some embodiments, the separation or detachment of the desired building fragment is achieved by a change in metal bond aspects of the walls of the reaction site. In some embodiments, the separation or detachment of the desired building fragment is achieved by a change in covalent bond aspects of the walls of the reaction site. In some embodiments, the separation or detachment of the desired building fragment is achieved by a change in ionic bond aspects of the walls of the reaction site.

Fragment Purification and/or Filtration and/or Q/C

As noted above, not every chemical reaction is 100% efficient, and the present system, as it is a multiplexed system, involves numerous reactions. As such, not every fragment in every well will necessarily be the exactly desired sequence (e.g., exact molecular weight to an accuracy of + or −1 amu (atomic mass unit) by mass spectrometry) for that fragment. This issue can be addressed in a number of ways. Of course, by reducing the size of each fragment, the number of reactions decreases and thus the probability of error decreases; thus, by keeping the size of the initial fragments shorter, this issue can be reduced.

Furthermore, in some embodiments, one can address this issue at the end of the process, by sequencing and/or selecting from a produced population of full length polymers to take the one desired sequence and to remove the others.

In some embodiments, one can include an additional process where one filters or purifies in the initial reaction site level products (prior to combining any of the fragments) by excluding those fragments that are shorter than the desired length of the fragment. As most errors will be due to an omission of the addition of a unit, this can be sufficient. Thus, a size exclusion filtering system can be employed in some embodiments

In some embodiments, the desired building fragment or first-level, second-level, fragment, subpolymer or desired polymer can be filtered by annealing to a complementary DNA microarray under very stringent conditions so that single base-pair mismatched fragments are washed away leaving desired DNA fragments available for further gene assembly.

In some embodiments, antibodies can be used as a screening method, binding to desired sequences or structures while not binding to undesired sequences or structures. In some embodiments, one or more of the above purification processes and/or structures can also (or alternatively) be used earlier in the process, such as following the separation of the desired building fragment from the surface. In some embodiments a Q/C level, apparatus, structure can also be included and can comprise a single molecule DNA sequencer. In some embodiments, this occurs at the final level or immediately before the output. In some embodiments, single and/or combinations of purifications techniques described herein, can be used before and after any and/or all combination sites.

### Light Source and Light Directing Device

In some embodiments, the device and/or method employs a light for photo generating a reagent (such as an acid) for the desired building fragment synthesis and/or a light for cleaving the desired building fragment from a desired reaction site. Various structures can be employed to achieve this. In some embodiments, each of these processes involves a different light source. One can emit wavelengths appropriate for cleavage (e.g., 300-400 nm, 190-600 nm) and the other can emit wavelengths appropriate for photodeprotection, photo coupling, photogeneration of a reagent for coupling, and photogeneration of reagent for cleavage (e.g., 190-600 nm). As will be appreciated by one of skill in the art, the specific light source employed will vary based upon the properties of the molecules used for photocleavage and photo generation; however, when both aspects are employed in one embodiment they will be appropriately matched. In some embodiments, more than one light source is employed (for example, one for each type of nucleic acid and one (or more) for photocleavage. In some embodiments, all of the light sources share the same light directing device (e.g., DLP) and in other embodiments, one or more light sources share one or more light directing device.

The light can be directed to specific reaction sites in a number of ways, for example DLPs will allow specific direction of on the scale that is appropriate for micronized systems. In some embodiments, a variety of lens and/or mirrors can be employed. In some embodiments, a filtering system is employed, such that a wider beam of light is employed, but light is filtered out unless it is desired for a specific reaction site. In some embodiments, reaction sites with integrated LEDs, or LASERs can be employed. In some embodiments, a single technique or combinations described herein may be used to direct light.

As will be appreciated by those in the art, other wavelengths of light can be included for the above processes, as long as they do not adversely impact the step being performed. Thus, if blue wavelengths of light do not have any photochemical activity in the photogeneration or cleavage, then the light source and light directing device can also transmit and direct such light. However, in some embodiments, light that results in cleavage should not be applied when photo generation of an acid for fragment growth is occurring (unless one wishes to both free and add to the fragment at the same time).

As will be appreciated by those of skill in the art, various filters can be used in order to select a subset of wavelengths to allow the desired wavelengths of light to pass through to the reaction site from the light source and/or light directing device. In some embodiments, the light directing device can comprise a filter. As will be appreciated by those of skill in the art, it is the final wavelength of light that enters the reaction site (after it leaves the light source and is directed by the light directing device, and passes through any optional filters) that will be appropriate for either photogeneration or photocleavage. How that end result is achieved can be varied in a number of ways, as noted above, for example, by altering the number of light sources, the wavelengths emitted by the light sources, the number of light directing devices, and wavelengths directed (or not directed) by the device, any filters positioned anywhere along the path, and even the optical properties of the device itself.

In some embodiments, the wavelengths of light are between 100 and 1000 nm. Some useful ranges for some embodiments are within wavelengths from 355 to 490 nm, 265-450 nm, and 190-500 nm. In some embodiments, lasers can be used to deliver a monochromatic light source (i.e., single wavelength without the need of filters). In some embodiments, lasers wavelengths may be chosen that fall within the activation spectrum for photogenerated reagents but not within the activation spectrum for photocleavage.

### Reaction Sites

The reaction sites can be made of a variety of materials and take on a variety of shapes and sizes. In some embodiments, the reaction sites provide for a reaction volume in which monomers can be assembled in relative isolation from monomer assembly in a different reaction site. As such, the structure of the reaction site is adequate to isolate monomer assembly in that reaction site from inadvertently causing monomer assembly in another reaction site. In some embodiments, this is achieved by having reaction sites in the form of a chamber or well, which are connected to the rest of the device via a flow path. Some “bleed over” of activated monomers or PGRs may occur as these active ingredients travel from one reaction site, via a flow path to another reaction site; however, the amount is insignificant for the purpose of the device. This can be further reduced if valves or gates are used. In some embodiments, the processes in the reaction sites occur in multiple rounds, so that any single dose of reagents will not meaningfully add to the fragment without the immediately following set of reagents (or energy conditions), thereby further limiting any inadvertent monomer addition in other wells.

In some embodiments, the sites include an input and an output flow path. In some embodiments, the input and output flow paths are the same flow path.

In some embodiments, the surface of the reaction site is made of Si, n-p-doped Si, other doped semiconductors, and non-doped semiconductors known in the art to be useful for MEMS and microfluidic device fabrication. In some embodiments, the surface allows for the attachment of a polymer fragment thereto, and thus the surface material can comprise SiO₂, glass, p-n-doped Si. semiconductors, doped semiconductors, conductors, insulators, gold, thiol compounds. transition metals, transition metal compounds, organic compounds, inorganic compounds, films, liquid crystal layers, biotin, strepavadin, proteins, antibodies, receptors, Langmuir-Blodget films, linkers, conducting polymers, enzymes, bio- and chemical catalysts, ionic surfaces, chelation surfaces, highly reactive functional groups, organic and inorganic polymers, fluorinated polymers, self-assembled superstructures, DNA probes, PNA probes, RNA probes, protein A, protein G, nucleic acid binding proteins, lectins, carbohydrates, lipid surfaces, and lipid bi-layer surfaces. The above surfaces can also be used for other parts of the device in some embodiments (such as the combination sites, flow paths, combination chambers, storage sites, etc.)

In some embodiments, additional material is added within a reaction site to thereby increase the surface area of the reaction site. In such embodiments, the material can include beads, rods, randomly shaped objects, so as to provide greater surface area and thus attachment points for the starting monomer which is to become the desired building fragment. In some embodiments, porous Si and porous versions of objects described herein alone and/or in combination may be used.

In some embodiments, the reaction site is open to the atmosphere. In some embodiments, the reaction site is sealed from the atmosphere. In some embodiments, at least one surface of the reaction site is adequately transparent to one or more wavelengths of light. In some embodiments, while at least one surface of the reaction site is transparent, other surfaces (such as one or more walls) are not transparent to light, so as to prevent or reduce any bleed through of light in one reaction site to another reaction site.

The number of reaction sites can range anywhere from 10 to 10,000,000, 100-1,000,000, 1,000-100,000, or 5,000-50,000, where approximately 10,000-50,000 is a typical number. The volume of the reaction sites can very and in some embodiments is between 1 aL to 10 mL, 100 fL to 1 mL, 1000 μL to 100 microliters, 100 μL to 1 microliter, 50 μL to 1 nL. In some embodiments, the number of rows can be from 1 to 1,000,000, for example at least 1, 10, 50, 100, 500, 1000, or more rows). In some embodiments, the number of columns can be from 1 to 1,000,000, for example at least 1, 10, 50, 100, 500, 1000, or more columns).

In some embodiments, the flow paths from the reaction sites (and/or the combination sites/chambers) collapse together in different numbers, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, 100 or more at a time, (including ranges defined as greater than any of the preceding and ranges defined between any two of the preceding) either together or into a single chamber together. In some embodiments, in order to have a greater degree of specificity in the polymer being created, a relatively smaller number of chambers, sites, or flow paths are collapsed together at each combination point. Thus, in some embodiments, no more than 2 to 10 flow paths collapse together into one combination chamber. In some embodiments, no more than 500 flow paths collapse together in to one chamber.

In some embodiments, the reaction site comprises a set of reagents adequate for the production of the desired building fragment. In some embodiments, the reaction site comprises a set of reagents adequate for synthesis of one or more desired building fragments. In some embodiments the ingredients include photo generated reagents. In some embodiments, the PGR includes one or more of a PGR acid, a PGR base, a PGR catalyst (i.e., photocaged catalyst), a PGR enzyme (i.e., photocaged enzyme), or other photo activatable ingredients that can be used to create a fragment. In some embodiments, this includes photo-caged reagents know in the art.

In some embodiments, the filled circles depicted in FIG. 1A-1C are optional. The filed circles are combination chambers and their function can also be achieved, for some embodiments, via flow paths and combinations thereof. In some embodiments, the rectangles in FIGS. 1A-1C are optional larger flow paths (70, 71, 270). In embodiments without the larger flow paths (70, 71, 270, etc), the combination sites themselves can be used to provide added volume to an area where solutions are combined.

In some embodiments, the reaction sites are between 1 aL to 1 kiloliter in volume. In some embodiments the preferred range is between 100 fL and 100 nL, In some embodiments, 1-50 pL can be used.

In some embodiments, the number of reaction sites can vary based upon the desired polymer created. In some embodiments, there are at least two reaction sites. In some embodiments, there are 2 to one million reaction sites.

In some embodiments, the initial monomer is attached to a surface of the reaction site by a method outlined in U.S. Pat. No. 6,426,184, entitled “Method and Apparatus for Chemical and Biochemical Reactions Using Photo-Generated Reagents”, herein incorporated by reference in its entirety.

In some embodiments, the reaction site comprises or includes an electrode surface, through which a bias can be applied to hold or release DNA from indexed reaction sites.

### Combination Sites and Combination Chambers

As noted above, the combination sites include any area that allows for combination of two or more desired building fragments. Thus, this can include the combination chamber itself (60 or 160), which can be a chamber that allows for more turbulence in the flow and thus more mixing, or simply allows for a greater volume of liquid and thus more combinations of samples can be added together, or simply serve as a combination point for two or more flow paths. This can also include flow paths that come together, and thereby allow for the combination of various products (e.g., 50, 51, 52, 70, 71, 251, 250, and 252).

In some embodiments, a heating element is associated with the sites or chambers. In some embodiments, entry or exit from the sites or chambers is controlled via valves. In some embodiments, inlet and/or outlet ports are provided in or proximal to the combination sites and/or chambers. In some embodiments, acoustic, magnetic and/or electrical field aspects of the walls of the combination site are used to control the heating of a solution or sample in the combination site.

In some embodiments, the walls of the combination chambers (and/or reaction site and/or flow path, and/or storage sites, etc.) are made of, for example, layers that are comprised of different materials including but not limited to the following: silicon based compounds (such as SiN, SiC, doped Si, etc), glass, PMMA, Parylene, elastomer, polymer or metal substrate. In some embodiments the reaction sites, flow paths, and combination sites are comprised of P+ Boron doped Silicon. In some embodiments the crystal orientation of the Si layer is <100>. In some embodiments the crystal orientation of the Si layer is <100>, <110>, <111>. In some embodiments the, sites, chambers, flow paths, and combination sites are comprised of 7740 Corning glass. In some embodiments, the material of the walls is adequately inert in regard to the fragments and ingredients to be used in the device so as to prevent any inadvertent sticking of the product to the walls.

In some embodiments, the combination sites and/or combination chambers are between 1 aL to 1 kiloliter, e.g., 2 to 1000 μL. In some embodiments the combination sites and/or combination chambers range in volume between 100 fL and 1 μL. In some embodiments, the combination sites and/or chambers are associated with a heating element or device that can adjust the temperature of the solution therein between 0 to 125 degrees Celsius over a 0 to 24 hour period. In some embodiments, the degree of temperature change and the extent of temperature change is such as to allow for the specific hybridization of two or more desired building fragments so as to form the desired first-level strand. In some embodiments, this can be achieved via heating and/or cooling the entire chip via a Peltier device or other molecular biology device used for controlling solution temperature during hybridization events.

In some embodiments, the number of combination sites and/or combination chambers can vary based upon the desired polymer created. In some embodiments, there are at least two combination sites and/or combination chambers. In some embodiments, there are 2 to one million combination sites and/or combination chambers. In some embodiments, 2 to 500, 5 to 100,000, 10 to 10,000, or 10 to 1000 combination sites and/or combination chambers are employed.

The number of combination sites that are serially related (that is, liquid flows from one combination site (e.g., a first-level combination site) or chamber into a second combination site (e.g., a second-level combination site), and thus levels of combination can vary for a particular chip or application. In some embodiments, there is at least one combination level, such that reaction sites are combined into one combination site. As depicted in FIG. 1A, there are numerous combination sites that are first-level combination sites (100-105), which are then combined into second-level combination site 200 (FIG. 1B), which is then combined into a third-level combination site 300, which, with other third-level combination sites, are combined together and stored in a storage site 1001. In some embodiments, the number of combination levels can be from at least 1 to 100 or more, e.g., 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, or 100 (including ranges defined as greater than any of the preceding values or between any two of the preceding values). In some embodiments, the number of combination levels can be from 1 to 20, for example 1, 2, 3, 4, 5, 6, 7, 8, 9, 10, 11, 12, 13, 14, 15, 16, 17, 18, 19, or 20.

In some embodiments, the degree of compression or combination that occurs at each combination site (and/or chamber) can be at least a combination of 2 flow paths from two different reaction sites and/or combination sites. In some embodiments, the combination is of 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, or 100 combination and/or reaction sites (including ranges defined as greater than any of the preceding values or between any two of the preceding values). In some embodiments, the combination is from 2 to 10 combination and/or reaction sites. In some embodiments, the combination is from 2 to 100 combination and/or reaction sites.

### Storage Sites

In some embodiments, the storage sites 1001-1006 can vary in number. The number of storage sites need not be limited and can be at least one. In some embodiments, there are at least 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, or storage sites (including ranges defined as greater than any of the preceding values or between any two of the preceding values. In some embodiments, there are 2 to 100 storage sites.

In some embodiments, the number of flow paths to the storage sites can vary. Thus, while in the embodiment in FIG. 1B, there are 6 flow paths 1010, 1011, 1012, 1013, 1014, and 1015, coming from flow path 316, which in turn comes from flow paths 310, 311, 312, 313, 314, and 315, in some embodiments there are more or fewer flow paths. In some embodiments, the flow paths can vary in length and size. In some embodiments, the flow paths are between 0.1 and 1000 microns in size. In some embodiments, the relationship between the size of the chamber (and flow path) and the distance down the flow system takes into account the conservation of volume as the flow paths are combined. In some embodiments, the flow paths are between 2-100 in number.

### Chip Fabrication

In some embodiments the device can be fabricated through a series of etching steps followed by bonding to seal the optically transparent cover layer with access ports over the layer containing the reaction sites, storage sites, combination sites, flow paths, etc. In some embodiments, these etching processes can generally involve photolithographic printing of reaction sites, flow paths, combination sites and any access ports to the inner workings of the device. In some embodiments the reaction sites, storage sites, flow paths, and/or combination sites are etched in a substrate and the access ports are etched in another. In some embodiments the reaction sites, flow paths, access ports and combination sites are etched into a silicon based compound, glass, PMMA, Parylene, elastomer, polymer or metal substrate. In some embodiments the reaction sites, flow paths, access ports and combination sites or combinations thereof are formed on opposite sides of the layers to be bonded. In some embodiments these layers are comprised of different materials including but not limited to the following: silicon based compounds (such as SiN, SiC, doped Si, etc), glass, PMMA, Parylene, elastomer, polymer or metal substrate. In some embodiments the reaction sites, flow paths, and combination sites are comprised of P+ Boron doped Silicon. In some embodiments the crystal orientation of the Si layer is <100>. In some embodiments the crystal orientation of the Si layer is <100>, <110>, <111>. In some embodiments the reaction sites, flow paths, and combination sites are comprised of 7740 Corning glass. In some embodiments these features can be created through precision machining such as milling or grinding. In some embodiments printing of the reaction sites, combination sites, flow paths, combination sites and access ports can be achieved through more than one photolithographic step. In some embodiments the first photolithographic step will be followed by an etching step. In some embodiments the first photolithographic step will be followed by an etching step and secondary photolithographic step followed by a second etch and so on. In other embodiments a sacrificial material may be used to limit etching in a particular direction or to achieve a specific channel aspect ratio or depth. In some embodiments access ports are created through a wet etch. In some embodiments the flow paths are created by a DRIE step. In some embodiments the reaction sites are created by a RIE step. In some embodiments the etching steps comprise one or more of the following: reactive ion etching (RIE), deep reactive ion etching (DRIE), plasma etching, wet etching through chemical solvents, ion milling and/or tool based milling. In some embodiments the bonding steps comprise one or more of the following: adhesive, creating covalent, noncovalent, hydrophobic, hydrogen bonds, plasma bonding, thermal bonding, welding, diffusion bonding, anodic bonding, fusion boding and/or eutectic bonding. In some embodiments the final chips are diced from a larger wafer containing many devices. However, as appreciated by those in the art, some details of the device fabrication will vary based on the intended use and desired properties of the device.

### Materials for the Device

In some embodiments, the device can be etched in Si, glass, polymer-based substrates such as PMMA, or metallic substrates or any other suitable substrate material and sealed with glass or other material to allow the adequate transmission of the appropriate wavelength of light to both build the desired building fragment and separate the desired building fragment. Of course, such properties only need be used for those areas that will be exposed to the light. Thus, in some embodiments, only the reaction wells are so configured. In some embodiments, the materials are compatible with DNA (ssDNA) (of course, not all embodiments require this). Other materials are provided above in regard to various sites and chambers and can be used

In some embodiments, the sealing layer can be comprised of quartz, borosilicate glass, PMMA, Parylene or any other material allowing for the passage of light for synthesis and cleavage of the ssDNA.

In some embodiment the chip is comprised of multiple layers with out of plane fluidic connections. These layers can be bonded or independently connected with fluid conduits.

In some embodiments, various deposition processes known in the art of MEMS microfluidic device fabrication such as chemical vapor deposition (CVD), PVD (plasma vapor deposition), electrochemical molecular layer epitaxy (EMOLE), underpotential deposition (UPD), molecular-beam epitaxy (MBE), atomic layer epitaxy, (ALE), metalloorganic molecular-beam epitaxy, (MOMBE), metallorganic chemical vapor deposition, (MOCVD), etc. are used to coat the surface of the reaction sites with a suitable attachment site for the first monomer of the desired polymer fragment.

### Flow Control

In some embodiments, the flow between the various flow paths and sites can be controlled. In some embodiments, this can be achieved via one or more valves that can obstruct, reduce, or completely block the flow of liquid from one section to another section. Positions of such valves include at each of the sites, and at the start, end or anywhere in-between for the flow paths. There can be a valve to control flow in or out of the site as well as a liquid inlet, allowing one to add liquid to the system. The valves can be selected from the following group: solenoid, ball, diaphragm, piston, needle, magnetic, pinch, thermal expansion/contraction, memory polymers, check valves or any other valve device capable of handling the reagents and pressures required for operation.

In some embodiments, the flow of a sample throughout the system can be controlled by controlling the flow of a liquid or gas into the system. This can force a sample through the various chambers, or effectively reduce the rate of movement through the various chambers. In some embodiments, the flow into the system is adjusted by adding liquid or gas, in other embodiments, the flow into the system is adjusted by removing or reversing the flow that had been added to the system. In some embodiments, the flow can be controlled by controlling the flow out of the system.

In some embodiments, the flow is relatively consistent, and control of fragment movement can be achieved, at least at the reaction site level, via optical manipulation (such as separation or detaching of a produced desired building fragment from a surface of the reaction site).

In some embodiments, one or more of the above options are combined. For example, in some embodiments, while the reaction sites 10 and 20 are not gated or valved with respect to the flow paths (50-52), valves can be present at or before one or more of the combination chambers, reaction sites, and one or more of the storage sites. Thus, in some embodiments, this will allow the coordinated addition of the various desired building fragments (or first-level, second-level, third-level, etc. fragments) together, as several can be let into the combination sites, the valves closed, and while the combination of the desired building fragments occur in the combination sites, further synthesis and release of further desired building fragments can occur upstream at the reaction site.

In some embodiments, the fluid control system is that described in U.S. Pat. Pub. 20040101444, herein incorporated by reference in its entirety. In some embodiments, the chip or device further comprises a pump or is connected to a pump and/or vacuum.

### Temperature Control

In some embodiments, one or more of the flow paths, reaction sites, combination sites, storage sites, purification areas, combination chambers, or any combination thereof includes or is associated with a device to allow one to control the temperature of the device from 0 to 125 degrees Celsius.

In some embodiments, the temperature control is through a programmable thermocycler known in the art. The thermocycler may be programmed to cycle between any temperatures from 0 to 125 degrees Celsius as a function of time. Specific temperature=f(time) equations specify temperature graphs (temperature ramps) that are crucial to the annealing and ligation process.

In some embodiments, this is achieved via a thermocycling element in the device or chip. In some embodiments, only a part of the chip or device is heated. In some embodiments, the heating is selective to one or more reaction sites, combination sites and/or combination chambers and/or flow paths and/or sites.

In some embodiments, the temperature of the solution in the chip is controlled by a resistive layer or heating element in the chip itself. The layer or heating element can be in the entire chip, or associated with various sites and/or chambers, and/or flow paths. In some embodiments, numerous heating layers or elements are provided and independently controllable. In some embodiments, a p-n doped Si arrangement, as used in a Peltier Cooling-heating device can be fabricated as part of the chip.

In some embodiments, the material of the chip aids in insolating the temperature of a solution in one site, flow path, or chamber from the temperature of a second solution in a second site, flow path, or chamber. In some embodiments, the material that makes up the combination sites or chambers, and the distance between neighboring sites or chambers, allows one to heat one well without adversely or unintentionally heating a proximal site or chamber.

In some embodiments, the chip or device is heated by an outside heat source. In some embodiments, the chip is heated by convection or direct contact with an external heat source.

In some embodiments, this can be achieved by focusing light energy on the chamber. In some embodiments, the light energy is selected so as to promote a superior heat delivery. In some embodiments, the light can be infrared light and can be directed via the light directing device to one or more of the chambers to be heated. In some embodiments, the light penetrates the outer surface of the device and enters the solution. In some embodiments, the light heats the outer surface and thus indirectly heats the solution inside. In some embodiments electromagnetic radiation is used to interact with elements in the chambers heating one or more of the chamber surfaces.

In some embodiments, the method and device noted herein allows for the simple low cost fabrication of such a device in a single layer without the need for complex components such as valves or multi layer bonded chip and impacts the manufacturability and cost of such a chip. In some embodiments, without the combination of the compression of the reaction sites into combination sites and then into a storage site, followed by the combination of products from numerous storage sites, the number of channels leaving the first level would make such a device unmanufacturable due to the large number of individually indexable valves required and/or the size.

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### Example 1

This example outlines how to use some of the embodiments disclosed herein for nucleic acid synthesis.

A device for the synthesis of DNA containing 7776 reaction sites is provided. A representation of this layout can be seen in FIGS. 1A and 1B. Shown in FIGS. 1A and 1B is the inlet in which A, C, G, T, U or any modified nucleic acid or nucleic acid analogue precursors (in the form of individual monomers, dimers (e.g., A-G), trimers (e.g., T-G-G), tetramers (e.g., T-C-U-G), pentamers, hexamers, and/or any combinations of precursors described herein) can be perfused as well as a buffer solution and ligase as the synthesis procedure demands. This inlet channel splits to feed all columns of the reaction site array.

The inlet has a switching valve to allow for different reactants to be added. In the schematic the represented chip has 108 rows by 72 columns of reaction sites for a total of 7776. In every column the 108 rows of reaction sites are collapsed 36 at a time into a single channel. FIG. 1A only shows the lower 40.

Linkage of nucleic acid monomers is achieved though the use of a photogenerated acid through individually addressable digital light processing (DLP) projection technology allowing individual reaction sites (shown in FIGS. 1A and 1B) to be addressed like pixels on a screen. Through this process a piece of single stranded DNA of any sequence and incremental length can be obtained. Cleavage of the ssDNA from the walls of the reaction sites is achieved by a photodegradable linkage molecule anchoring the ssDNA to the surface of the reaction sites. The wavelength of light used in synthesis does not interfere with the wavelength of light used in cleavage. This combination of indexable photoacid generation followed by an indexable photocleavage allows DNA synthesis to occur without the use of on chip valves.

A ligase suspended in a buffer solution is then perfused through the 7776 reaction sites which collapse 36 at a time into 216 individual channels. These channels are combined 6 at a time into assembly chambers where ssDNA is assembled into double stranded DNA (dsDNA).

Once enough fluid has been added to displace the cleaved ssDNA into the assembly chambers the chip undergoes a temperature cycle to anneal the DNA pieces together. At the first level of combination, 6 pieces of ssDNA combine to form a single piece of dsDNA three linear strands or increments long (i.e., three forward and three reverse strands or increments). The subsequent assembly levels then bring pieces of dsDNA together for ligation forming pieces of dsDNA 18, 108 and 648 linear increments long. After the dsDNA reaches 648 DNA linear increments in length, given a 50 base long ssDNA increment, the length of the dsDNA is approximately 32,400 base pairs long. This length of dsDNA is on the order of most viral genomes ranging from 10 kbps to 50 kbps.

After the DNA contents have been combined they are stored in 1 of 6 channels in a storage site. This area of the chip is isolated from the temperature cycling on the previous combination sites and held at less than 310 K until at least two of the storage spaces are full. The DNA pieces are driven into these parking spaces through an off chip valving system. By opening the valve controlling the port of DNA storage site, fluid can be perfused though the open port pushing the DNA into P1 (storage site 1) while the ports controlling P2 through P6 and the outlet are closed. For the next assembly run P1 is closed and the outlet is opened and the process repeats however on this pass after the DNA has been combined, instead P2 is opened while P1, P3 through P6 and the outlet are closed. Once all the storage sites are full, buffer is perfused through the ports on P1 though P6 and ligase is added through the port directly upstream of the parking area pushing the contents of P1 through P6 into the final combination site/chamber to be ligated. The result is a dsDNA assembly, given a 50 base long ssDNA increment, of 194 kbps. This is the size of a minimal genome operating system in one on chip synthesis.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

### EQUIVALENTS

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A method for synthesis of a desired polymer comprising:
(a) providing a first reaction site comprising a first monomer attached to a surface of the reaction site;
(b) selectively irradiating the first reaction site, thereby coupling an additional monomer to the first monomer;
(c) repeating said irradiating until a desired building fragment has been synthesized, wherein the desired building fragment is created while the first monomer is attached to the surface;
(d) separating the desired building fragment from the surface;
(e) repeating, processes a through d as desired to create a desired number of desired building fragments;
(f) combining the first desired building fragment with a second desired building fragment so as to create a first subpolymer;
(g) storing the first subpolymer in a first storage site;
(h) repeating processes (a) through (f) so as to make a second subpolymer and storing said second subpolymer in a second storage site; and
(i) combining the first and second subpolymers so to form a desired polymer.

2. The method of claim 1, wherein the irradiating process results in a photo generated reagent and wherein the polymer comprises a nucleic acid.

3. The method of claim 2, wherein the nucleic acid comprises a DNA.

4. The method of claim 1, wherein process (d) involves irradiating the desired building fragment with a wavelength of light that disrupts the attachment of the first monomer to the surface of the reaction site without inadvertently adding an additional monomer to the desired building fragment.

6. The method of claim 1, wherein the first reaction site comprises a chamber that is fluidly isolated from the second reaction site.

7. The method of claim 1, wherein process (f) comprises combining the first desired building fragment with the second desired building fragment so as to create a first-level fragment.

8. The method of claim 7, wherein the first-level fragment comprises a double stranded nucleic acid, a multi stranded fragment, or a double stranded and a multistranded fragment.

9. The method of claim 1, wherein process (f) comprises combining i) the first desired building fragment with ii) the second desired building fragment, and iii) a third desired building fragment to form a first-level fragment.

10. The method of claim 9, wherein the first-level fragment is chemical or enzyme ligated forming a first ligated first-level fragment.

11. The method of claim 10, wherein the process of claims 9 and 10 are repeated so as to form a second ligated first-level fragment, and wherein the first and second ligated first-level fragments are combined so as to allow the first and second ligated first-level fragments to hybridize to one another, forming a second-level fragment.

12. The method of claim 11, further comprising the process of performing a ligation reaction thereby forming a ligated second-level fragment.

13. The method of claim 12, further comprising combining the one or more ligated second-level fragments to create the subpolymer.

14. The method of claim 1, wherein the desired building fragment comprises a random nucleotide sequence.

15. The method of claim 1, wherein selectively irradiating the first reaction site, thereby coupling a monomer to the first monomer comprises a wavelength in the 100 to 1000 nm range.

16. The method of claim 1, wherein process (d) involves a wavelength in the 100 to 1000 nm range, wherein the wavelength in process (d) does not overlap with the wavelength in processes (b) and/or (c).

17. The method of claim 1, further comprising one or more filtering processes so that an undesired building fragment that has been synthesized in a reaction site is not contained within the subpolymer.

18. The method of claim 1, further comprising a process of purification so that an undesired final polymer that has been synthesized in combination sites is not contained within the final gene synthesis product.

20. A device for parallel and serial polymer creation comprising:
a first reaction site;
a second reaction site, wherein the first and second reaction sites comprise a surface that allows for the attachment of a polymer to said surface in each reaction site, wherein the at least two reaction sites are effectively optically transparent to a first set of wavelengths of light that allows for the creation of a photo generated reagent, wherein the first and second reaction sites are effectively transparent to a second set of wavelengths of light that allows for the cleavage of a bond that connects a nucleic acid to said surface;
a first-level combination sites, wherein the first and second reaction sites are in fluid communication with the first-level combination site, wherein the fluid communication allows for the combination of a sample from the first reaction site with a sample from a second reaction site, wherein the first-level combination site is associated with a heating element that controls the temperature of the first-level combination site so as to control nucleic acid annealing; and
one or more storage sites, controllably fluidly connected to the first-level combination site, wherein the one or more storage sites can be controllably fluidly sealed from the first-level combination site.

21. The device of claim 20, further comprising a nucleic acid that is at least 10,000 nucleotides in length.

22. The device of claim 18, further comprising a light directing apparatus for selectively directing light to the first reaction site while avoiding directing light to the second reaction site.

23. The device of claim 20, wherein the reaction site comprises one or more of SiO₂, glass, p-n-doped Si. semiconductors, doped semiconductors, conductors, insulators, gold, thiol compounds. transition metals, transition metal compounds, organic compounds, inorganic compounds, films, liquid crystal layers, biotin, strepavadin, proteins, antibodies, receptors, Langmuir-Blodget films, linkers, conducting polymers, enzymes, bio- and chemical catalysts, ionic surfaces, chelation surface, highly reactive functional groups, organic and inorganic polymers, fluorinated polymers, self-assembled superstructures, DNA probes, PNA probes, RNA probes, protein A, protein G, nucleic acid binding proteins, lectins, carbohydrates, lipid surfaces, or a lipid bi-layer surface.

24. The device of claim 20, wherein first-level combination site comprises a channel that connects the at least two reaction sites to one another.

25. The device of claim 20, wherein the device comprises:
at least 7776 reaction sites;
at least 216 first-level combination sites;
at least 36 second-level combination sites;
at least 6 third level combination sites; and
at least 6 storage sites.

26. A device for polymer synthesis comprising:
a first reaction site and a second reaction site, fluidly connected to a combination site, wherein the combination site is fluidly connected to a first storage site;
a third reaction site and a fourth reaction site, fluidly connected to a combination site, wherein the combination site is fluidly connected to a second storage site;
a second combination site, fluidly connected to the first storage site and the second storage site; and
an output, wherein the output allows a fluid in the second combination site to exit the device.

27. A device for polymer synthesis comprising:
a first reaction site and a second reaction site, fluidly connected to a purification site, followed by a combination site, wherein the combination site is fluidly connected to a storage site;
a third reaction site and a fourth reaction site, fluidly connected to a purification site, followed by a combination site, wherein the combination site is fluidly connected to a second storage site;
a second combination site, fluidly connected to the first storage site and the second storage site;
an output, wherein the output allows a fluid in the second combination site to enter a final purification site and then exit the device.

28. The device of claim 27, further comprising a purification site which is a size exclusion filtering system.
